(19) United States
(12) Patent Application Publication
(10) Pub. No.: US 2009/0270511 A1
(43) Pub. Date: Oct. 29, 2009

(54) PRODRUGS OF INHIBITORS OF CATHEPSIN S

(75) Inventor: Jacques Yves Gauthier, Laval (CA)

Correspondence Address: MERCK AND CO., INC, P O BOX 2000, RAHWAY, NJ, 07065-0907, US

(73) Assignee: MERCK FROSST CANADA LTD.

(21) Appl. No.: 12/439,323

(22) PCT Filed: Sep. 7, 2007
(86) PCT No.: PCT/CA07/01584

§ 371 Date: Feb. 27, 2009

Related U.S. Application Data
(60) Provisional application No. 60/843,054, filed on Sep. 8, 2006.

(51) Int. Cl. A61K 31/165; C07C 255/46

(52) U.S. Cl. 514/618; 558/393

(57) ABSTRACT

The present invention provides compounds of formula (I) which are prodrugs of inhibitors of cathepsin S and as such are useful in the prevention and treatment of cathepsin S dependent diseases and conditions including, but not limited to, chronic obstructive pulmonary disease (COPD) and pain.

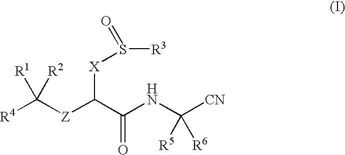

### BACKGROUND OF THE INVENTION

Cathepsin S is a cysteine protease that belongs to the papain superfamily. It is most highly expressed in lung followed by lymph nodes, spleen, ileum, adipose, liver, heart and microglial of the brain. Cathepsin S has a restricted cell type distribution; it is expressed in antigen presenting cells such as B cells, dendritic cells, macrophage as well as smooth muscle cells and tumour cells. It is found in the type II alveolar cells and the resident macrophages of the lung. It resides intracellularly in acidic endosomes/lysosomes and is also secreted extracellularly where it is presumed to function at or near the cell surface. It has been documented to be regulated by IFNγ, LPS and proinflammatory cytokines such as TNFα or IL-1β. The neurotrophic factors, bFGF and NGF have been shown to increase expression and activity of Cat S. As well, in vivo, the transgenic overexpression of IL-13 leads to increased expression of Cat S and increased lung volume, mucus and inflammation, hallmarks of an emphysematous COPD phenotype. Cathespin S has diverse endopeptidase, di-peptidyl-peptidase and aminopeptidase activities. It has broad substrate activity against such proteins as the MHC class II invariant chain (Ii), MBP, SLPI, DPP1, amyloid precursor protein, amyloid beta peptide and insulin, as well as activity against extracellular matrix proteins such as elastin, collagen, fibronectin, laminin and heparan sulfate. Cystatins are endogenous tight-binding inhibitors of Cathepsin S.

Cathepsin S (abbreviated Cat S) is implicated in Alzheimer's disease, Down's syndrome, atherosclerosis, chronic obstructive pulmonary disease, cancer, osteoarthritis, Gaucher disease, myoclonus epilepsy (EPMI) and certain autoimmune disorders, including, but not limited to juvenile onset diabetes, multiple sclerosis, pemphigus vulgaris, Graves' disease, myasthenia gravis, systemic lupus erythematosus, rheumatoid arthritis and Hashimoto's thyroiditis; allergic disorders, including, but not limited to asthma; and allogenic immune responses, including, but not limited to, rejection of organ transplants or tissue grafts, see (C. A Lemere et al., Am J. Pathol 146: 848-860, 1995; J. S. Munger et al., Biochem J 1995 311, 299-305; J. Liu et al., Arterioscler Thromb Vasc Biol 24: 1359-1366, 2004; G. K. Sukhova et al., J Clin Invest 2003 111, 897-906; T. Flannery et al., Am J Pathol 163: 175-182, 2003; P. L. Fernandez et al., Int J Cancer 95: 51-55, 2001; M. Soderstrom et al., Matrix Biol 19: 717-725, 2001; M. T. Moran et al., Blood 96: 1969-1978, 2000; R. Rinne et al., Ann Med 34: 380-385, 2002; H. Yang et al., J Immunol 174: 1729-1737, 2005; N. Cimerman et al., Pflugers Arch 442: R204-206, 2001; T. Zheng et al., J Clin Invest 2000 106, 1081-93; G. P. Shi et al., Circ Res 2003 92, 493-500; T. Y. Nakagawa et al., Immunity 1999 10, 207-17).

The levels of Cat S mRNA have been found to be significantly increased in the brains of Creutzfeldt-Jakob disease patients (C. A. Baker et al., J Virol 76: 10905-10913, 2002; F. Dandoy-Dron et al., JBC 273: 7691-7697, 1998). Due to its high elastinolytic activity, it has also been suggested that cathepsin S is involved in vascular matrix remodeling during angiogenesis and the promotion of cilia motility in the lung. Increased Cathepsin S levels have been found in the extracellular environment during various pathological conditions, such as, tumor invasion, atherogenesis and muscular dystrophy. Cathepsin S inhibitors have been shown to inhibit other disorders such as atherosclerosis and Th1 type inflammation. Cathepsin S knock out mice and inhibitor studies show a clear role for the intracellular Cat S in MHC class II invariant chain processing whereby it cleaves the invariant chain (Ii) p10 fragment to allow peptide exchange in the class II peptide binding groove. Thus, Cat S is the limiting step in antigen presentation. Complete knock-down of Cat S levels demonstrated that high fractional inhibition of Cat S is required before immune responses in the mouse are modulated, while data obtained from Cat S heterozygotic mice showed no effect on Ii degradation. Cathepsin S may also play a role in antigen processing. More recently, increased cathepsin S mRNA was found in animal models of chronic pain. It was demonstrated that inhibition of Cat S with a small molecule inhibitor reversed the mechanical hyperalgesia in these animals (PCT Application WO 03/020287).

The crystal structure of cathepsin S with and without inhibitors has been resolved. Also, selective inhibitors of cathepsin S have been reported in, for example, D. J. Gustin et al., Bioorg & Med Chem Lett, 15: 1687-1691, 2005; R. L. Thurond et al., J Med Chem, 47: 4799-4801, 2004; V. Leroy and S. Thurairatnam, Expert Opin. Ther. Patents, 14: 301-311, 2004; R. L. Thurmond et al, J Pharmacol Exp Ther., 308:268-276, 2004; N. Katunuma et al., Biol Chem, 384: 883-890, 2003; C. L. Cywin et al., Bioorg Med Chem, 11: 733-740, 2003; N. E. Zhou et al., Bioorg Med Chem, 13: 139-141, 2003; K. Saegusa et al., J Clin Invest, 110: 361-369, 2002; Y. D. Ward et al., J Med Chem., 45:5471-5482, 2002; B. Walker et al., Biochem Biophys Res Commun, 275:401-405, 2000; N. Katunuma FEBS Lett, 458: 6-10, 1999; D. Bromme et al., Biol Chem Hoppe Seyler, 375: 343-347, 1994). Cathepsin S inhibitors would be useful in treating disorders involving inflammation and tissue remodeling; allogenic, autoimmune, neurological or allergic disorders; cancer; as well as inflammatory or neuropathic pain.

PCT Application WO2005/028429 discloses cathepsin S inhibitors of the formula:

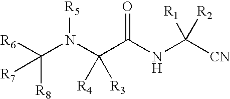

### SUMMARY OF THE INVENTION

The present invention relates to prodrugs of potent and selective inhibitors of cathepsin S, which are useful in the treatment and prevention of various cathepsin S dependent diseases and conditions. The present invention also relates to methods for using the inhibitors in the prevention and treatment of cathepsin S dependent diseases and conditions as well as pharmaceutical compositions containing the inhibitors.

### DETAILED DESCRIPTION OF THE INVENTION

The present invention provides compounds of formula I and pharmaceutically acceptable salts thereof:

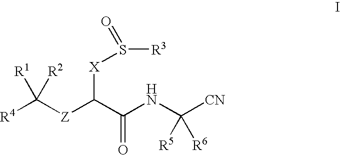

wherein

### X is —(CHR^{b})n;

### Z is NH, O, S or CH₂;

n is an integer selected from 1 to 6;
R¹ is C₁₋₆ haloalkyl, aryl, heteroaryl, aryl-C₁₋₆alkyl-, or heteroaryl-C₁₋₆alkyl- wherein said aryl and heteroaryl are optionally substituted with 1 to 3 substituents independently selected from C₁₋₆alkyl, C₁₋₆alkoxy, halo, C₁₋₆ haloalkyl, C₃₋₆cycloalkyl, C₁₋₆ haloalkoxy, —SR^{a}, —S(O)R^{a}, —S(O)₂R^{a}, —OR^{a}, NR^{b}R^{c}, cyano, and aryl;
R² is hydrogen or C₁₋₆ haloalkyl;
R³ is C₁₋₆alkyl, C₁₋₆ haloalkyl, C₃₋₆cycloalkyl, C₃₋₆cycloalkyl-C₁₋₆alkyl-, aryl, aryl-C₁₋₆alkyl-, heteroaryl, or heteroaryl-C₁₋₆alkyl-, wherein cycloalkyl is optionally substituted with C₁₋₃ haloalkyl, and wherein aryl and heteroaryl are optionally substituted with 1 to 3 substituents independently selected from C₁₋₆alkyl, halo, C₁₋₆ haloalkyl, C₃₋₆cycloalkyl, C₁₋₆ haloalkoxy, —SR^{a}, —S(O)₂R^{a}, —S(O)₂R^{a}, —OR^{a}, NR^{b}R^{c}, cyano, and aryl;
R⁴ is C₁₋₆alkyl, C₁₋₆ haloalkyl, C₃₋₆cycloalkyl, C₃₋₆cycloalkyl-C₁₋₆alkyl-, Ar¹, Ar¹—C₁₋₆alkyl-, Ar¹—Ar², or Ar¹—Ar²—C₁₋₆alkyl-, wherein Ar¹ and Ar² are independently selected from optionally substituted aryl and optionally substituted heteroaryl wherein the optional substituents are 1 to 3 groups independently selected from C₁₋₆alkyl, CH(OH)C₁₋₆alkyl, C₂₋₆ alkenyl, halo, C₁₋₆ haloalkyl, CH(OH)C₁₋₆ haloalkyl, C₃₋₆cycloalkyl, C₁₋₆ haloalkoxy, —SR^{a}, —S(O)R^{a}, —S(O)₂R^{a}, —S(O)₂NR^{b}R^{c}, —OR^{a}, NR^{b}R^{c}, nitro, cyano, heterocyclyl, —C(O)OR^{a}, —C(O)R^{a}, —C(O)NR^{b}R^{c}, —NR^{b}CONR^{b}S(O)₂R^{a}, —OSO₂R^{a}, —N(R^{b})C(O)NR^{b}R^{c}, —N(R^{b})C(O)R^{a}, —N(R^{b})C(O)OR^{a}, —N(R^{b})SO₂R^{a}, —C(R^{a})(R^{b})NR^{b}C(R^{a})(R^{b}), —C(R^{a})(R^{b})C(R^{a})(R^{b})NR^{b}R^{c}, —C(O)C(R^{a})(R^{b})NR^{b}R^{c}, and C(R^{a})(R^{b})C(O)NR^{b}R^{c};
R⁵ and R⁶ are independently selected from hydrogen, C₁₋₆ alkyl and C₂₋₆ alkenyl wherein said alkyl and alkenyl groups are optionally substituted with 1 to 6 halo, C₃₋₆cycloalkyl, —SR^{a}, S(O)R^{a}, S(O)₂R^{a}, OR^{a}, or NR^{b}R^{c};
or R⁵ and R⁶ together with the carbon atom to which they are attached form a C₃₋₈ cycloalkyl ring or a heterocyclyl ring wherein said ring system is optionally substituted with C₁₋₆ alkyl or halo;
R^{a} is hydrogen, C₁₋₆alkyl, aryl, heteroaryl, aryl-C₁₋₆alkyl and heteroaryl-C₁₋₆alkyl;
R^{b} and R^{c} are independently hydrogen or C₁₋₆alkyl; or
R^{b} and R^{c}, when attached to a nitrogen atom, together complete a 4- to 6-membered ring optionally having a second heteroatom selected from O, S and N—R^{d}; and
R^{d} is hydrogen or C₁₋₆alkyl.

In one subset of formula (I) are compounds wherein R¹ is C₁₋₆ haloalkyl, and R² is hydrogen. In one embodiment R¹ is trifluoromethyl.

In another subset of formula (I) are compounds wherein R⁵ and R⁶ are independently selected from hydrogen and C₁₋₆ alkyl. In one embodiment R⁵ and R⁶ are each hydrogen; in another embodiment one of R⁵ and R⁶ is hydrogen and the other is methyl; in another embodiment R⁵ and R⁶ are each methyl.

In another subset of formula (I) are compounds wherein R⁵ and R⁶ together with the carbon atom to which they are attached form a C₃₋₈ cycloalkyl ring wherein said ring is optionally substituted with C₁₋₆ alkyl or halo. In one embodiment thereof R⁵ and R⁶ together with the carbon atom to which they are attached form a cyclopropyl ring.

In another subset of formula (I) are compounds wherein X is —(CH₂)ₙ— where n is an integer of from 1 to 3. In one embodiment thereof X is —CH₂—; in another embodiment thereof X is —CH₂CH₂—.

In another subset of formula (I) are compounds wherein Z is selected from —NH— and —O—. In one embodiment thereof Z is —NH—.

In another subset of formula (I) are compounds wherein R³ is selected from C₁₋₆alkyl, C₁₋₆ haloalkyl, aryl, and aryl-C₁₋₆alkyl-, wherein aryl is optionally substituted with 1 to 3 substituents independently selected from C₁₋₆alkyl, halo, and C₁₋₆ haloalkyl. In one embodiment thereof, R³ is C₁₋₆ alkyl, optionally substituted phenyl or optionally substituted benzyl, wherein the substituents are 1 to 3 halo atoms. In another embodiment thereof R³ is selected from methyl, 2,3-difluorobenzyl, and 2-methylpropyl.

In another subset of formula (I) are compounds wherein R⁴ is optionally substituted Ar¹ or optionally substituted —Ar¹—Ar² wherein the substituents are 1 to 3 groups independently selected from halo, —CH(OH)C₁₋₆alkyl, C₁₋₆ haloalkyl and CH(OH)C₁₋₆ haloalkyl. In one embodiment thereof R⁴ is phenyl or biphenyl each of which is optionally substituted with 1 to 2 halo atoms, —CH(OH)CHF₂ or —CH(OH)CF₃. In another embodiment thereof R⁴ is bromophenyl or fluorophenyl.

In another subset of formula (I) are compounds of formula (Ia) having the stereoconfiguration as shown and pharmaceutically acceptable salts thereof:

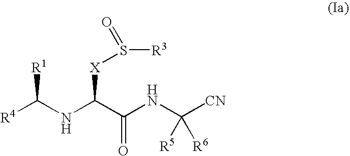

wherein X, R¹, R³, R⁴, R⁵ and R⁶ are as defined under formula (I). In one subset of formula (Ia), X is —CH₂— or —CH₂CH₂—. In another subset of formula (Ia) R¹ is trifluoromethyl. In another subset of formula (Ia) R⁴ is phenyl or biphenyl each optionally substituted with 1 or 2 halo atoms, or with the group —CH(OH)CHF₂ or —CH(OH)CF₃. In yet another subset of formula (Ia), R⁵ and R⁶ together with the carbon atom to which they are attached form a cyclopropyl ring.

In another subset of formula (I) are compounds of formula (Ib) and pharmaceutically acceptable salts thereof:

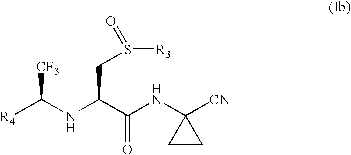

wherein R³ is selected from C₁₋₆alkyl, C₁₋₆ haloalkyl, aryl, and aryl-C₁₋₆alkyl-, wherein aryl is optionally substituted with 1 to 3 substituents independently selected from C₁₋₆alkyl, halo, and C₁₋₆ haloalkyl; and R⁴ is optionally substituted Ar¹ wherein the substituents are 1 to 3 groups independently selected from halo, —CH(OH)C₁₋₆alkyl, C₁₋₆ haloalkyl and CH(OH)C₁₋₆ haloalkyl. In one embodiment of formula (Ib), R³ is C₁₋₆ alkyl. In another embodiment of formula (Ib), R⁴ is phenyl optionally substituted with one or two halogen atoms.

Unless otherwise stated, the following terms have the meanings indicated below:

“Alkyl” as well as other groups having the prefix “alk” such as, for example, alkoxy, alkanoyl, and the like, means carbon chains which may be linear or branched or combinations thereof. Examples of alkyl groups include methyl, ethyl, propyl, isopropyl, butyl, sec- and tert-butyl, pentyl, hexyl and the like.

“Alkenyl” means carbon chains which may be linear or branched or combinations thereof containing at least 1 carbon to carbon double bond. Examples of alkenyl groups include ethenyl, 1-propenyl, 2-propenyl, 2-methyl-1-propenyl, 1-butenyl and 1-hexenyl.

“Aryl” means any stable monocyclic or bicyclic carbon ring of up to 10 atoms wherein at least one ring is aromatic carbocycle. In cases where the aryl substituent is bicyclic and the second ring is non-aromatic (e.g., cycloalkyl, cycloalkenyl, heterocyclyl), it is understood that attachment is via the aromatic ring. Examples of aryl group include phenyl, naphthyl, tetrahydronaphthyl, methylenedioxyphenyl, 1,2,3,4-tetrahydroquinolin-5-yl, 4- or 5-indanyl, and 4- or 5-indenyl.

“Cycloalkyl” means carbocycles containing no heteroatoms, and includes mono- and bicyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spiro-fused ring systems. In cases where the cycloalkyl substituent is bicyclic and the second ring is aryl, heteroaryl or heterocyclyl, it is understood that attachment is via the non-aromatic carbocyclic ring. Examples of cycloalkyl include cyclopropyl, cyclobutyl, cyclopentyl, cyclohexyl, decahydronaphthalene, adamantane, indanyl, indenyl, 1,2,3,4-tetrahydronaphthalene and the like.

“Haloalkyl” means an alkyl radical as defined above wherein at least one and up to all of the hydrogen atoms are replaced with a halogen. Examples of such haloalkyl radicals include chloromethyl, 1-bromoethyl, fluoromethyl, difluoromethyl, trifluoromethyl, 2,2,2-trifluoroethyl and the like.

“Halogen” or “halo” means fluorine, chlorine, bromine and iodine.

“Heteroaryl” means a stable monocyclic or bicyclic ring of up to 10 atoms wherein at least one ring is aromatic and contains from 1 to 4 heteroatoms selected from the group consisting of O, N and S. Heteroaryl groups within the scope of this definition include, but are not limited to, pyrrolyl, imidazolyl, pyrazolyl, pyridyl, pyrimidinyl, pyrazinyl, pyridazinyl, furanyl, thienyl, oxazolyl, isoxazolyl, oxadiazolyl, thiazolyl, isothiazolyl, thiadiazolyl, triazolyl, tetrazolyl, indolyl, isoindolyl, benzimidazolyl, benzofuranyl, benzothienyl, benzofurazanyl, benzopyrazolyl, benzotriazolyl, benzoxazolyl, benzothiazolyl, benzisoxazolyl, benzisothiazolyl, quinolinyl, isoquinolinyl, cinnolinyl, quinazolinyl, quinoxalinyl, indolinyl, indolazinyl, indazolyl, isobenzofuranyl, naphthyridinyl, tetrazolopyridyl, dihydrobenzoimidazolyl, dihydrobenzofuranyl, dihydrobenzothiophenyl, dihydrobenzoxazolyl, dihydroindolyl, dihydroquinolinyl, tetrahydroquinolinyl. In cases where the heteroaryl substituent is bicyclic and one ring is non-aromatic (e.g, cycloalkyl, cycloalkenyl or heterocyclyl), it is understood that attachment is via the heteroaromatic ring; if both rings are aromatic and one contains no heteroatom, the attachment can be via either ring. If the heteroaryl contains nitrogen atoms, it is understood that the corresponding N-oxides thereof are also encompassed by this definition.

“Heterocyclyl” means a 5- to 10-membered mono- or bicyclic nonaromatic ring containing from 1 to 4 heteroatoms selected from the group consisting of O, N and S. In cases where the heterocyclyl substituent is bicyclic the second ring may be aryl, heteroaryl, heterocyclyl, cycloalkyl or cycloakenyl; in such case it is understood that attachment is via the heterocyclic ring. “Heterocyclyl” includes, but is not limited to the following: piperazinyl, piperidinyl, pyrrolidinyl, morpholinyl, thiomorpholinyl, tetrahydropyranyl, tetrahydrothiophenyl and the like. If the heterocycle contains a nitrogen, it is understood that the corresponding N-oxides thereof are also encompassed by this definition.

### Optical Isomers—Diastereomers—Geometric Isomers—Tautomers.

Compounds described herein contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereo-chemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

### Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

### Pharmaceutical Compositions.

Another aspect of the present invention provides pharmaceutical compositions which comprise a compound of Formula I, or a pharmaceutically acceptable salt thereof, and a pharmaceutically acceptable carrier. The term “composition”, as in pharmaceutical composition, is intended to encompass a product comprising a compound of formula (I), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

The pharmaceutical compositions of the present invention comprise a compound represented by Formula I (or pharmaceutically acceptable salts thereof), a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the compound of the present invention is being administered. The pharmaceutical compositions may be conveniently presented in unit dosage form and prepared by any of the methods well known in the art of pharmacy.

In practice, the compounds represented by Formula I, or pharmaceutically acceptable salts thereof, of this invention can be combined in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of a compound of formula (I). Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association a compound of the present invention with the carrier that constitutes one or more necessary ingredients. In general, the compositions are prepared by uniformly and intimately admixing a compound of formula (I) with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, a compound of formula (I) in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of a compound of formula (I), and each cachet or capsule preferably containing from about 0.1 mg to about 500 mg of a compound of formula (I).

Pharmaceutical compositions of the present invention suitable for parenteral administration may be prepared as solutions or suspensions of a compound of formula (I) in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

Pharmaceutical compositions for administration by inhalation or insufflation may be formulated for delivery in the form of an aerosol spray from pressurized packs or nebulizers. They may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons, and dry powder inhalation (DPI) aerosol, which may be formulated as a dry powder of a compound of Formula I with or without additional excipients. A dry powder composition, for example a powder mix of a compound of formula (I) and a suitable carrier such as lactose, may be presented in unit dosage form in, for example, capsules, cartridges or blister packs from which the powder may be administered with the aid of an inhaler. Examples of dry powder inhalers that may be suitable for use with the present compositions may be found in Newman, S. P., Expert Opin. Biol. Ther., 2004, 4(1):23-33.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The following are examples of representative pharmaceutical dosage forms for the compounds of Formula I:

| Inj. Suspension (I.M.) | mg/mL | Tablet | mg/tab. | Capsule | mg/cap. |
|---|---|---|---|---|---|
| Cmpd of Formula I | 10 | Cmpd of Formula I | 25 | Cmpd of Formula I | 25 |
| Methylcellulose | 5.0 | Microcryst. Cellulose | 415 | Lactose Powder | 573.5 |
| Tween 80 | 0.5 | Povidone | 14.0 | Magnesium Stearate | 1.5 |
| Benzyl alcohol | 9.0 | Pregelatinized Starch | 43.5 | | 600 |
| Benzalkonium chloride | 1.0 | Magnesium Stearate | 2.5 | | |

| | |
|---|---|
| Water for injection to a total | 500 |
| volume of 1 mL | |

### Utilities

Compounds of this invention are prodrugs of potent and selective inhibitors of cathepsin S, and as such are useful in the treatment and prevention of cathepsin S dependent diseases and conditions in mammals, preferably human. Thus another aspect of the present invention provides a method for the prevention or treatment of cathepsin S dependent diseases and conditions in a mammal which comprises administering to said mammal a therapeutically effective amount of a compound of formula (I). This aspect encompasses the use of a compound of formula (I) for the manufacture of medicament for the treatment or prevention of cathepsin S dependent diseases and conditions.

Compounds of the present invention are prodrugs of corresponding sulfones of formula (II):

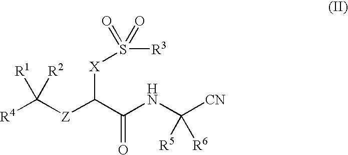

Unexpectedly, compounds of the present invention, unlike their corresponding sulfones, display linear pharmacokinetics; they are significantly better absorbed than the sulfones, and are efficiently converted in vivo to the sulfones. Significantly higher levels of the sulfones are measured in the blood and tissues of living organisms when these are dosed with sulfoxides, compared to dosing with sulfones.

Cathepsin S dependent diseases and conditions which compounds of formula (I) may be useful in the treatment or prevention include, but are not limited to, Alzheimer's disease, Down's syndrome; atherosclerosis and myocardial infarct and stroke, chronic obstructive pulmonary disease including emphesyma and chronic bronchitis, cancer, osteoarthritis, Gaucher Disease, myoclonus epilepsy, and certain autoimmune disorders, including but not limited to, juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders including but are not limited to rejection of organ transplants or tissue grafts; and pain including visceral pain (such as pancreatitis, interstitial cystitis, renal colic, prostatitis, chronic pelvic pain), neuropathic pain (such as postherpetic neuralgia, acute zoster pain, nerve injury, the “dynias”, e.g., vulvodynia, phantom limb pain, root avulsions, radiculopathy, painful traumatic mononeuropathy, painful entrapment neuropathy, carpal tunnel syndrome, ulnar neuropathy, tarsal tunnel syndrome, painful diabetic neuropathy, painful polyneuropathy, trigeminal neuralgia), central pain syndromes (potentially caused by virtually any lesion at any level of the nervous system including but not limited to stroke, multiple sclerosis, spinal cord injury), and postsurgical pain syndromes (eg, postmastectomy syndrome, postthoracotomy syndrome, stump pain)), bone and joint pain (osteoarthritis), spine pain (e.g., acute and chronic low back pain, neck pain, spinal stenosis), shoulder pain, repetitive motion pain, dental pain, sore throat, cancer pain, myofascial pain (muscular injury, fibromyalgia), postoperative, perioperative pain and preemptive analgesia (including but not limited to general surgery, orthopedic, and gynecological), chronic pain, dysmenorrhea (primary and secondary), as well as pain associated with angina, and inflammatory pain of varied origins (e.g. osteoarthritis, rheumatoid arthritis, rheumatic disease, teno-synovitis and gout, ankylosing spondylitis, bursitis).

### Dose Ranges

The magnitude of prophylactic or therapeutic dose of a compound of Formula I will vary with the nature and severity of the condition to be treated, and with the particular compound of Formula I used and its route of administration. The dose will also vary according to the age, weight and response of the individual patient. In general, the daily dose range lies within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0.1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

For the treatment or prevention of COPD, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be administered as a single daily dose or divided for twice or thrice daily administration.

For the treatment or prevention of pain, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be administered as a single daily dose or divided for twice or thrice daily administration.

For the treatment of rheumatoid arthritis, a compound of Formula I may be used at a dose of from about 0.1 mg/kg to about 100 mg/kg, preferably from about 1 mg/kg to 10 mg/kg, by oral/inhalation/sublingual/etc. once, twice, three times daily, etc. The dose may be administered as a single daily dose or divided for twice or thrice daily administration.

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) morphine and other opiate receptor agonists including propoxyphene (Darvon) and tramadol; (2) non-steroidal antiinflammatory drugs (NSAIDs) including COX-2 inhibitors such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and the coxibs (celecoxib, valecoxib, rofecoxib and etoricoxib); (3) corticosteroids such as betamethasone, budesonide, cortisone, dexamethasone, hydrocortisone, methylprednisolone, prednisolone, prednisone and triamcinolone; (4) histamine H1 receptor antagonists such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, desloratadine, fexofenadine and levocetirizine; (5) histamine H2 receptor antagonists such as cimetidine, famotidine and ranitidine; (6) proton pump inhibitors such as omeprazole, pantoprazole and esomeprazole; (7) leukotriene antagonists and 5-lipoxygenase inhibitors such as zafirlukast, montelukast, pranlukast and zileuton; (8) drugs used for angina, myocardial ischemia including nitrates such as nitroglycerin and isosorbide nitrates, beta blockers such as atenolol, metoprolol, propranolol, acebutolol, betaxolol, bisoprolol, carteolol, labetalol, nadolol, oxprenolol, penbutolol, pindolol, sotalol and timolol, and calcium channel blockers such as diltiazam, verapamil, nifedipine, bepridil, felodipine, flunarizine, isradipine, nicardipine and nimodipine; (9) incontinence medications such as antimuscarinics, e.g., tolterodine and oxybutinin); (10) gastrointestinal antispasmodics (such as atropine, scopolamine, dicyclomine, antimuscarinics, as well as diphenoxylate); skeletal muscle relaxants (cyclobenzaprine, carisoprodol, chlorphenesin, chlorzoxazone, metaxalone, methocarbamol, baclofen, dantrolene, diazepam, or orphenadrine); (11) gout medications such as allopurinol, probenicid and colchicine; (12) drugs for rheumatoid arthritis such as methotrexate, auranofin, aurothioglucose and gold sodium thiomalate; (13) drugs for osteoporosis such as alendronate and raloxifene; decongestants such as pseudoephedrine and phenylpropanolamine; (14) local anesthetics; (15) anti-herpes drugs such as acyclovir, valacyclovir and famcyclovir; (16) anti-emetics such as ondansetron and granisetron; (17) migraine drugs such as the triptans (e.g. rizatriptan, sumatriptan), ergotamine, dihydroergotamine, CGRP antagonists, antidepressants (e.g., tricyclic anti-depressants, serotonin-selective reuptake inhibitors, beta-adrenergic blockers); (18) VR1 antagonsits; (19) anticonvulsants (e.g., gabapentin, pregabalin, lamotrigine, topiramate, carbamazepine, oxcarbazepine, phenyloin); (20) glutamate antagonists (e.g., ketamine and other NMDA antagonists, NR2B antagonists); (21) acetaminophen; (22) CCR2 antagonists; (23) PDE4 antagonists; (24) muscarinic M3 receptor antagonists such as tiotropium; (25) HMG-CoA reductase inhibitors such as lovastatin, simvastatin, atorvastatin, fluvastatin, pravastatin, and cerivastatin; (26) bradykinin B1 receptor antagonists.

### Biological Activity

### In Vitro Assays

Recombinant human Cat S was from Calbiochem, while recombinant human Cat L was from R&D Systems. Human liver Cat B was from Sigma. Pre-pro-form humanized rabbit Cathepsin K (rabbit cathepsin K with S163A, Y175D and V274L mutations introduced; numbered from initial methionine) was expressed in and purified from the media fraction of Hek 293 cells, then acid activated. All protease substrates were from Bachem.

Enzyme activity assays: Assays of Cat S were carried out in 50 mM MES pH 6.5, 100 mM NaCl, 2.5 mM DTT, 2.5 mM EDTA, 0.001% w/v BSA, 10% DMSO and 40 μM Z-Val-Val-Arg-AMC as substrate. Assays of Cat B were carried out in 50 mM MES pH 6.0, 2.5 mM DTT, 2.5 mM EDTA, 0.001% Tween-20, 10% DMSO and 83 μM Boc-Leu-Lys-Arg-AMC as substrate. Assays of humanized rabbit Cat K and Cat L were carried out in 50 mM MES pH 5.5, 2.5 mM DTT, 2.5 mM EDTA, 10% DMSO and 2 μM Z-Leu-Arg-AMC as substrate. Prior to the addition of substrate, inhibitor (10.0 μM to 0.02 nM) was pre-incubated for 2 min with each enzyme (0.1-11 nM) to allow the establishment of the enzyme-inhibitor complex. Substrate was then added and the enzyme activity measured from the increase of fluorescence at 460 nm (λₑₓ=355 nm). Assays were performed in 96-well plate format and the plate read using a Gemini EM (Molecular Devices) plate reader. The substrate concentrations employed represent Kₘ or sub-Kₘ values. The percent inhibition of the reaction was calculated from a control reaction containing only vehicle. IC₅₀ curves were generated by fitting percent inhibition values to a four parameter logistic model (SoftmaxPro, Molecular Devices). Compounds of formula (I) generally have IC₅₀ values of about 1 μM or lower.

### In Vivo Neuropathic Pain Model

Mice (C5B16, Taconic) were anesthetized with 2% gaseous isoflurane. An incision was made just below the hip bone, parallel to the sciatic nerve. The nerve was exposed, and any adhering tissue removed from the nerve. A tight ligature with 6-0 silk suture thread around ⅓ to ½ of the diameter of the sciatic nerve was made. Muscles were closed with suture thread and the wound with wound clips. The response of the mice to mechanical stimulation was tested before and 4 days after nerve injury.

Animals were placed in plastic cages with a wire mesh floor and allowed to acclimate for 15-45 min before each test session. Mechanical sensitivity was determined with calibrated von Frey filaments using the up-and-down paradigm (Chaplan, et al. (1994) J. Neurosci. Methods 53, 55-63). The von Frey filaments were applied to the mid-plantar surface for 8 s or until a withdrawal response occurred. Following a positive response, an incrementally weaker stimulus was tested. If there was no response to a stimulus, then an incrementally stronger stimulus was presented. After the initial threshold crossing, this procedure was repeated for four stimulus presentations per animal per test session. Mechanical sensitivity was then assessed at various times post oral administration of the test compound (2 to 24 hours). Percent reversal of allodynia was calculated as: (post-drug−post-surgery)/(pre-surgery−post-surgery)×100, where 100% is equivalent to complete reversal of allodynia, i.e. pre-surgery value.

### Methods

The following schemes and descriptions are provided to illustrate processes for the preparation of compounds of formula (I) and intermediates therefor. Those skilled in the art will readily understand that known variations of the conditions and processes of the following preparative procedures can be used. In the following schemes PG represents a protecting group for a reactive functional group such as amine, hydroxy and carboxyl groups, and LG represents a leaving group. The selection of a protecting group, its introduction and subsequent removal are well known to those skilled in the art and can be found in standard texts such as Greene and Wuts, Protective Groups in Organic Synthesis, 3^{rd} Edition, 1999 (Wiley Interscience). Similarly, the selection and use of a leaving group in a displacement reaction is well known to a person skilled in the art, and are discussed in standard organic chemistry textbooks such as March, Advanced Organic Chemistry, 5^{th} Edition, 2001 (Wiley Interscience).

### Abbreviations Used

The following abbreviations have the meanings indicated, unless stated otherwise in the specification: DIPEA=N,N-diisopropylethylamine; DMF=dimethylformamide; eq.=equivalent(s); ES (or ESI)— MS=electron spray ionization−mass spectroscopy; Et=ethyl; EtOAc=ethyl acetate; HATU=O-(7-azabenzotriazol-1-yl)-N,N,N′,N′-tetramethyluronium hexafluorophosphate; MTBE=methyl t-butyl ether; MeOH=methanol; NMR=nuclear magnetic resonance; RT=Room temperature.

Compounds of the present invention may be prepared as depicted in the following scheme:

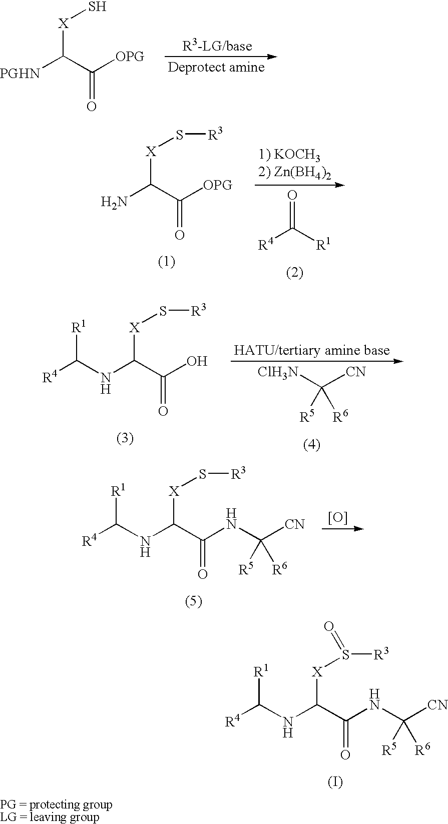

Amino- and carboxy-protected cysteine is treated with a base such as potassium carbonate and an R³-LG in DMF followed by deprotection of the resulting derivative to provide the amine (1). Amine (1) and a ketone (2) are treated with a base such as potassium methoxide in a solvent such as methanol. The product is treated in situ at low temperature (−40° C.) with a reducing agent such as zinc borohydride to yield the carboxylic acid (3). This acid is then coupled to the aminoacetonitrile derivative (4) with a coupling agent such as HATU and a base such as DIPEA to provide the sulfanyl derivative (5). Oxidation of the (5) to the corresponding sulfinyl derivative (I) is accomplished using an oxidizing agent such as m-chloroperoxybenzoic acid in dichloromethane, magnesium monoperoxyphthalate in methanol or hydrogen peroxide in combination with sodium tungstate and a phase transfer reagent such as tetrabutylammonium hydrogen sulfate in ethyl acetate.

The following examples are provided to illustrate the invention and are not to be construed as limiting the scope of the claims in any manner.

### Reference Example 1

### N²-[(1S)-1-(4-bromophenyl)-2,2,2-trifluoroethyl]-N¹-(1-cyanocyclopropyl)-3-(methylsulfonyl)-L-alaninamide

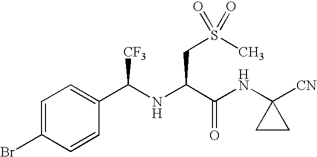

Step 1. To a −5° C. solution of methyl N-(tert-butoxycarbonyl)-L-cysteinate (45.8 g, 195 mmol) in DMF (600 mL, 0.325M) was added iodomethane (13.38 mL, 214 mmol, 1.1 eq) followed by potassium carbonate (27 g, 195 mmol, 1 eq) and the mixture was stirred overnight at 5° C. It was poured over water and little aqueous NH₄Cl and extracted with Et₂O (3×) washed with dilute NaHCO₃ and brine, dried and stripped to dryness to yield methyl N-(tert-butoxycarbonyl)-S-methyl-L-cysteinate (48.1 g), which was used as such in the next step.
Step 2. Acetyl chloride (13.72 mL, 193 mmol, 1 eq) was added slowly to −15° C. methanol (50 mL, 3.86M) and the mixture was reacted for 1 hr. The product of Step 1 (48.1 g, 193 mmol), as a methanol (20 mL) solution, was then added and the mixture was reacted at 20° C. for 4 hrs. It was evaporated to dryness and swished in MTBE (500 mL) at 20° C., then dried on high vacuum overnight to give methyl S-methyl-L-cysteinate HCl. ¹H NMR (CD₃OD) δ: 4.3-4.4 (1H, m), 3.9 (3H, s), 3.0-3.2 (2H, m), 2.2 (3H, s).
Step 3. To a −78° C. suspension of 1-(4-bromophenyl)-2,2,2-trifluoroethanone (17.5 g, 69.2 mmol) and methyl S-methyl-L-cysteinate HCl (14.85 g, 80 mmol, 1.15 eq) in MeOH (75 mL) was added potassium methoxide, 95% (10.2 g, 138 mmol, 2 eq) and the mixture was allowed to warm to RT and was stirred overnight. To this suspension, cooled to −40° C., was added CH₃CN (400 mL) and then a zinc borohydride suspension (prepared by adding sodium borohydride (10.48 g, 277 mmol, 4 eq) portion-wise to a 0° C. suspension of zinc chloride (18.81 g, 138 mmol, 2 eq) in glyme (150 mL) and stirring overnight at RT) was transferred slowly, over 30 min. at −40° C. The mixture was stirred for 2 hrs and then acetone (150 mL) was added dropwise. The mixture was then allowed to warm to RT. It was poured on ice, water and EtOAc and the pH adjusted to c.a. 5 with 1N HCl. It was extracted twice with EtOAc, washed with brine and dried. The mixture was purified on SiO2 using 1:3 EtOAc and hexanes followed by 1:3 EtOAc and hexanes containing 10% acetic acid to yield a mixture of N-[(1S)-1-(4-bromophenyl)-2,2,2-trifluoroethyl]-S-methyl-L-cysteine and N-[(1R)-1-(4-bromophenyl)-2,2,2-trifluoroethyl]-S-methyl-L-cysteine (12.4 g), which was used as such in Step 4. ¹H NMR (CD₃OD) δ: 7.6-7.7 (2H, d), 7.5 (2H, d), 4.55-4.65 (1H, m), 4.6-4.7 (1H, m), 2.85-2.95 (2H, m), 2.15 (3H, s).
Step 4. To a −5° C. solution of the mixture from Step 3 (12.4 g, 33.3 mmol), HATU (18.97 g, 49.9 mmol, 1.5 eq) and 1-amino-1-cyclopropanecarbonitrile-HCl (5.92 g, 49.9 mmol, 1.5 eq) in DMF (50 mL, 0.666 M) was added DIPEA (34.9 mL, 200 mmol, 6 eq) dropwise and the mixture was reacted at 0° C. for 1.5 hr. It was poured in ice and water and then extracted twice with 1:1 EtOAc and diethyl ether. The combined organic layers were washed with water, brine and dried. Chromatography on SiO2 using 1:3 EtOAc and hexanes followed by 1:2 EtOAc and hexanes yielded N²-[(1S)-1-(4-bromophenyl)-2,2,2-trifluoroethyl]-N¹-(1-cyanocyclopropyl)-S-methyl-L-cysteinamide and N²-[(1S)-1-(4-bromophenyl)-2,2,2-trifluoroethyl]-N¹-(1-cyanocyclopropyl)-S-methyl-L-cysteinamide (11.2 g), MS (+ESI): 436.2, 438.0 [M+1]⁺
Step 5. To a 0° C. suspension of the mixture from Step 4 (11.2 g, 25.7 mmol) in EtOAc (250 mL, 0.103M) was added sodium tungstate dihydrate (102 mg, 0.308 mmol, 0.012 eq) and tetrabutylammonium hydrogen sulfate (445 mg, 1.311 mmol, 0.051 eq). To this was added hydrogen peroxide 30% (6.43 mL, 62.9 mmol, 2.449 eq) dropwise and the mixture was allowed to warm to RT and was stirred for 4 hrs. The mixture was diluted with EtOAc and washed with dilute aqueous sodium thiosulfate and brine. It was purified by chromatography on SiO₂ using 1:25 MeOH:CH₂Cl₂. The residue after evaporation was triturated in MTBE for 16 hours. Filtration and drying yielded the title compound (9.6 g). ¹H NMR (CD₃COCD₃) δ 8.5 (1H, bs), 7.6-7.7 (2H, d), 7.4-7.5 (2H, d), 4.45-4.55 (1H, m), 3.75-3.75 (1H, m), 3.5-3.6 (1H, m), 3.25-3.4 (2H, m), 3.15 (3H, s), 1.4-1.5 (2H, m), 1.05-1.25 (2H, m).

### Reference Example 2

### N¹-(1-Cyanocyclopropyl)-3-(methylsulfonyl)-N²-[(1S)-2,2,2-trifluoro-1-(4-fluorophenyl)ethyl]-L-alaninamide

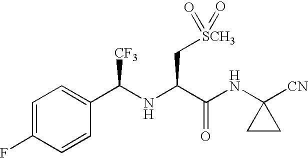

Step 1. Following the procedure of Reference Example 1, Step 3, but using 2,2,2,4′-tetrafluoro-acetophenone and methyl S-methyl-L-cysteinate HCl, S-methyl-N-[(1S)-2,2,2-trifluoro-1-(4-fluorophenyl)ethyl]-L-cysteine was prepared, which was used as such in Step 2. ¹H NMR (CD₃COCD₃) δ: 7.55-7.65 (2H, m), 7.15-7.25 (2H, m), 4.55-4.65 (1H, m), 3.6-3.7 (1H, m), 2.8-3.0 (3H, hidden under water), 2.15 (3H, s).
Step 2. Following the procedure of Reference Example 1, Step 4, using the compound of above Step 1N¹-(1-cyanocyclopropyl)-S-methyl-N²-[(1S)-2,2,2-trifluoro-1-(4-fluorophenyl)ethyl]-L-cysteinamide was prepared. ¹H NMR (CD₃COCD₃) δ: 8.3 (1H, NH), 7.55-7.65 (2H, m), 7.25-7.35 (2H, m), 4.5 (1H, m), 3.35-3.45 (1H, m), 3.0 (1H, NH), 2.8-2.9 (2H, m), 2.1 (3H, s), 1.35-1.45 (2H, m), 1.0-1.2 (2H, m).
Step 3. Following the procedure of Example 1, Step 5 using the compound of above Step 2, the title compound was prepared. MS (+ESI): 407.9 [M+1]+.

### Example 1

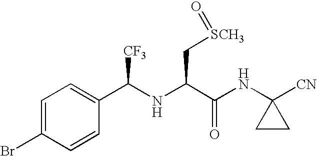

### N²-[(1S)-1-(4-bromophenyl)-2,2,2-trifluoroethyl]-N¹-(1-cyanocyclopropyl)-3-(methylsulfinyl)-L-alaninamide

To a 0° C. suspension of the compound of Reference Example 1, Step 4 (1.73 g, 3.97 mmol) in EtOAc (26.5 mL, 0.15 M) was added sodium tungstate dihydrate (39.3 mg, 0.119 mmol, 0.03 eq) and tetrabutylammonium hydrogen sulfate (67.6 mg, 0.199 mmol, 0.05 eq). To this was added hydrogen peroxide 30% (385 μL, 3.77 mmol, 0.95 eq) dropwise and the mixture was allowed to warm to RT and was stirred for 16 hrs. The mixture was washed with dilute aqueous sodium thiosulfate and brine. It was then passed on a short plug of SiO₂ using 5% ethanol in ethyl acetate. The residue was swished in MTBE for 1 hr to provide the title compound (1.05 g). NMR showed also about 1.2:1 of isomeric sulfoxides. MH+1 (+ESI) 451.8, 453.8

### Example 2

### N¹-(1-Cyanocyclopropyl)-3-(methylsulfinyl)-N²-[(1S)-2,2,2-trifluoro-1-(4-fluorophenyl)ethyl]-L-alaninamide

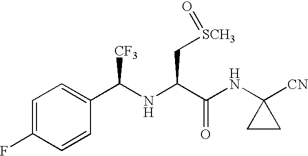

To a solution of the compound of Reference Example 2 (52.4 g, 140 mmol) in dichloromethane-methanol (70 mL, 1:1, 0.102M) at 0° C. was added magnesium monoperoxyphthalate hexahydrate (43.3 g, 70 mmol, 0.5 eq). The reaction mixture was stirred at 0° C. for 3 h, poured to an icy saturated NaHCO₃ solution and extracted with CH₂Cl₂ (2×80 mL). The combined organic layers were washed with a saturated NaCl solution, dried (MgSO₄) and concentrated under vacuum. The residue was purified by chromatography on silica gel (EtOH/CH₂Cl₂, 4:96) followed by recrystallization in ethyl acetate/hexanes to afford the title compound (44 g). MS (+EST) 392.2.

### Example 3

Male Sprague-Dawley rats (sex and strain may vary as specified by the investigator) are fasted overnight for each per os (PO) blood level. Test compound is prepared at a standard dose volume of 10 ml/kg by suspending the appropriate amount (mg/kg) of the test compound in an appropriate vehicle such as PEG 200/400, Methocel 0.5%-1.0%, sterile water/dextrose, Tween 80, solutions in vegetable oils, or 25% Molecusol. Sodium dodecyl sulphate (SDS) at concentration ranging from 0.04-1%, but preferably at 0.2%, can be added to some of the vehicle above, in particular to 0.5% methocel. Suspension of the test compound is administered by gavage, and blood samples are taken at 0, 15 min, 30 min, 1 h, 2 h, 4 h, 6 h, and 24 hr post drug administration via tail vein. The rats can be given food once the 4 h blood sample has been taken. Water must be provided at all times during the study. The plasma samples (typically 100 uL) are mixed with acetonitrile (typically 200 uL), vortexed and centrifuged. The supernatant is then analysed by LC-MS (liquid chromatography-mass spectrometry) to determine the concentration of the test compound, or if a sulfoxide is the test compound, the concentration of the corresponding sulfone. The results, concentration vs sampling time, are plotted. AUC is defined as the area under the curve.

| | |
|---|---|
| Dose: | AUC* |

Test Compound = Compound of Example 1
(sulfoxide dosed in SDS/methocel; sulfone AUC reported)

| | |
|---|---|
| 5 mg/kg | 43 |
| 10 mg/kg | 87 |
| 50 mg/kg | 484 |

Test Compound = Compound of Reference Example 1
(sulfone dosed in SDS/methocel; sulfone AUC reported)

| | |
|---|---|
| 5 mg/kg | 43 |
| 25 mg/kg | 59 |
| 50 mg/kg | 111 |

*AUC is area under the curve plotting plasma levels of the sulfone vs. sampling time.

1. A compound of formula I and pharmaceutically acceptable salts thereof: 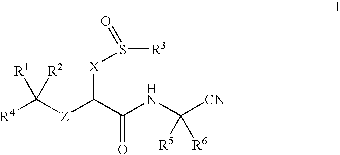
wherein
X is —(CHR^{b})n;
Z is NH, O, S or CH₂;
n is an integer selected from 1 to 6;
R¹ is C₁₋₆ haloalkyl, aryl, heteroaryl, aryl-C₁₋₆alkyl-, or heteroaryl-C₁₋₆alkyl- wherein said aryl and heteroaryl are optionally substituted with 1 to 3 substituents independently selected from C₁₋₆alkyl, C₁₋₆alkoxy, halo, C₁₋₆ haloalkyl, C₃₋₆cycloalkyl, C₁₋₆ haloalkoxy, —SR^{a}, —S(O)R^{a}, —S(O)₂R^{a}, —OR^{a}, NR^{b}R^{c}, cyano, and aryl;
R² is hydrogen or C₁₋₆ haloalkyl;
R³ is C₁₋₆alkyl, C₁₋₆ haloalkyl, C₃₋₆cycloalkyl, C₃₋₆cycloalkyl-C₁₋₆alkyl-, aryl, aryl-C₁₋₆alkyl-, heteroaryl, or heteroaryl-C₁₋₆alkyl-, wherein cycloalkyl is optionally substituted with C₁₋₃ haloalkyl, and wherein aryl and heteroaryl are optionally substituted with 1 to 3 substituents independently selected from C₁₋₆alkyl, halo, C₁₋₆ haloalkyl, C₃₋₆cycloalkyl, C₁₋₆ haloalkoxy, —SR^{a}, —S(O)R^{a}, —S(O)₂R^{a}, —OR^{a}, NR^{b}R^{c}, cyano, and aryl;
R⁴ is C₁₋₆alkyl, C₁₋₆ haloalkyl, C₃₋₆cycloalkyl, C₃₋₆cycloalkyl-C₁₋₆alkyl-, Ar¹, Ar¹—C₁₋₆alkyl-, Ar¹—Ar², or Ar¹—Ar²—C₁₋₆alkyl-, wherein Ar¹ and Ar² are independently selected from optionally substituted aryl and optionally substituted heteroaryl wherein the optional substituents are 1 to 3 groups independently selected from C₁₋₆alkyl, CH(OH)C₁₋₆alkyl, C₂₋₆ alkenyl, halo, C₁₋₆ haloalkyl, CH(OH)C₁₋₆ haloalkyl, C₃₋₆cycloalkyl, C₁₋₆ haloalkoxy, —SR^{a}, —S(O)R^{a}, —S(O)₂R^{a}, —S(O)₂NR^{b}R^{c}, —OR^{a}, NR^{b}R^{c}, nitro, cyano, heterocyclyl, —C(O)OR^{a}, —C(O)R^{a}, —C(O)NR^{b}R^{c}, —NR^{b}CONR^{b}S(O)₂R^{a}, —OSO₂R^{a}, —N(R^{b})C(O)NR^{b}R^{c}, —N(R^{b})C(O)R^{a}, —N(R^{b})C(O)OR^{a}, —N(R^{b})SO₂R^{a}, —C(R^{a})(R^{b})NR^{b}C(R^{a})(R^{b}), —C(R^{a})(R^{b})C(R^{a})(R^{b})NR^{b}R^{c}, —C(O)C(R^{a})(R^{b})NR^{b}R^{c}, and C(R^{a})(R^{b})C(O)NR^{b}R^{c};
R⁵ and R⁶ are independently selected from hydrogen, C₁₋₆ alkyl and C₂₋₆ alkenyl wherein said alkyl and alkenyl groups are optionally substituted with 1 to 6 halo, C₃₋₆cycloalkyl, —SR^{a}, S(O)R^{a}, S(O)₂R^{a}, OR^{a}, or NR^{b}R^{c};
or R⁵ and R⁶ together with the carbon atom to which they are attached form a C₃₋₈ cycloalkyl ring or a heterocyclyl ring wherein said ring system is optionally substituted with C₁₋₆ alkyl or halo;
R^{a} is hydrogen, C₁₋₆alkyl, aryl, heteroaryl, aryl-C₁₋₆alkyl and heteroaryl-C₁₋₆alkyl;
R^{b} and R^{c} are independently hydrogen or C₁₋₆alkyl; or
R^{b} and R^{c}, when attached to a nitrogen atom, together complete a 4- to 6-membered ring optionally having a second heteroatom selected from O, S and N—R^{d}; and
R^{d} is hydrogen or C₁₋₆alkyl.

2. A compound of claim 1 wherein R¹ is C₁₋₆ haloalkyl and R² is hydrogen.

3. A compound of claim 1 wherein R⁵ and R⁶ together with the carbon atom to which they are attached form a C₃₋₈ cycloalkyl ring wherein said ring is optionally substituted with C₁₋₆ alkyl or halo.

4. A compound of claim 1 wherein X is —(CH₂)ₙ— where n is an integer of from 1 to 3.

5. A compound of claim 1 wherein Z is —NH—.

6. A compound of claim 1 wherein R³ is selected from C₁₋₆alkyl, C₁₋₆ haloalkyl, aryl, and aryl-C₁₋₆alkyl-, wherein aryl is optionally substituted with 1 to 3 substituents independently selected from C₁₋₆alkyl, halo, and C₁₋₆ haloalkyl.

7. A compound of claim 1 wherein R⁴ is optionally substituted Ar¹or optionally substituted —Ar¹—Ar² wherein the substituents are 1 to 3 groups independently selected from halo, CH(OH)C₁₋₆alkyl, C₁₋₆ haloalkyl and CH(OH)C₁₋₆ haloalkyl.

8. A compound of claim 1 having the formula (Ia) and pharmaceutically acceptable salts thereof: 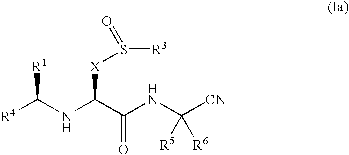
wherein X, R¹, R³, R⁴, R⁵ and R⁶ are as defined in claim 1.

9. A compound of claim 1 having the formula (Ib) and pharmaceutically acceptable salts thereof: 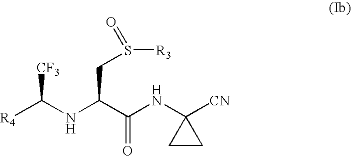
wherein R³ is selected from C₁₋₆alkyl, C₁₋₆ haloalkyl, aryl, and aryl-C₁₋₆alkyl-, wherein aryl is optionally substituted with 1 to 3 substituents independently selected from C₁₋₆alkyl, halo, and C₁₋₆ haloalkyl; and R⁴ is optionally substituted Ar¹ wherein the substituents are 1 to 3 groups independently selected from halo, —CH(OH)C₁₋₆alkyl, C₁₋₆ haloalkyl and CH(OH)C₁₋₆ haloalkyl.

10. A pharmaceutical composition comprising a compound of claim 1 or a pharmaceutically acceptable salt thereof and a pharmaceutically acceptable carrier.

11. (canceled)

12. A method for the prevention or treatment of diseases and conditions mediated by cathepsin S comprising administering to a patient in need thereof a therapeutically effective amount of a compound of claim 1.
